(19) United States
(12) Patent Application Publication
(10) Pub. No.: US 2011/0087239 A1
(43) Pub. Date: Apr. 14, 2011

(54) Device for Hygienic Extraction of an Acne Related Impaction

(76) Inventor: Thomas Bradford Howell, Kensington, MD (US)

(21) Appl. No.: 12/577,441

(22) Filed: Oct. 12, 2009

(51) Int. Cl. A61B 17/00; A45D 44/00

(52) U.S. Cl. 606/131

(57) ABSTRACT

The invention relates to a finger-assisted device for hygienic extraction of an acne related skin impaction, and methods and kits for hygienic extraction of acne related skin impaction.

### TECHNICAL FIELD

The invention relates to a finger-assisted device for hygienic extraction of acne related skin impactions, and methods and kits for hygienic extraction of acne related skin impaction.

### BACKGROUND OF THE INVENTION

Acne is a common skin condition that develops as a result of blockages in hair follicles. The most common form of acne is known as “acne vulgaris”, meaning “common acne”. One of the earliest signs associated with these blockages or impactions of the follicles is hyperkeratinization and formation of a plug of keratin and sebum (a microcomedo). Enlargement of sebaceous glands and an increase in sebum production occur with increased androgen production. The microcomedo may enlarge to form an open comedo (or blackhead) or closed comedo (whitehead). Whiteheads typically occur as a result of skin pores becoming clogged with sebum, a naturally occurring oil, and dead skin cells. In many instances, the naturally occurring largely commensal bacteria Propionibacterium acnes can cause inflammation, leading to inflammatory lesions (including papules, infected pustules, or nodules) in the dermis around the microcomedo or comedo. These inflammatory lesions typically result in redness and may also result in scarring or hyperpigmentation.

People who suffer from acne pimples whiteheads and blackheads consider them unsightly, painful, and depressing. People desire their removal and are frequently compelled to remove them, in the worst instances, using whichever sharp, poking, tweezing or cutting tool is readily available, frequently causing infection and permanent scarring. To a less extreme, people attempt the extraction using their fingers or fingernails, an unsanitary, often ineffective method that frequently damages the skin and is unsanitary in many regards including the potential for bacteria from under fingernails to enter the newly ruptured skin. Abrasions are caused by fingers and fingernails gauging and “skidding” across the skins surface as a result of losing traction on oily or moist skin when “pushing” a pimple. Infections are also caused as bacteria from under one's fingernails, or from dirty implements enter the newly ruptured skin. Moreover, acne bacteria, unless killed, causes acne pimples, which can lead to terrible scarring that is often permanent.

In addition to scarring and infections, other common effects of acne are psychological, including reduced self-esteem and, in some instances, more serious psychological effects including depression. Acne often appears during adolescence, when people already tend to be most socially insecure. Early treatment is therefore advocated to lessen the overall impact to individuals as much as possible.

Many previous attempts have been made to treat acne, although they all have major problems and shortcomings. One example of a conventional device that has been utilized in the past is a “comedo extractor.” The comedo extractor is not disposable and thus re-use of the extractor increases the chance of infection unless time-consuming and laborious sanitizing efforts are taken to clean the instrument. However, proper sanitizing is a requirement that is usually overlooked by its user. Among its many problems and disadvantages, the comedo extractor does not adjust to different sized impactions requiring the user the expense of purchasing multiple units of varying sizes. The comedo extractor is also not capable of drawing in tissue underneath the impaction to lift it, and relies instead on a depressing action of the tool from above the impaction that causes infection and scarring if too much force is applied. This shortcoming has led the manufacturers to recommend an invasive and aggressive lancing or puncture of the skin that produces scarring, is painful, and hazardous to the user. The typical comedo extractor has a razor-sharp lancet attached to the end of the tool on the opposite side of the extractor, thereby making the tool dangerous when it is sharp, and ineffective when the blade becomes dull. Moreover, a comedo extractor without the lancet burdens its user with sterilization and hazardous waste disposal of sharp, contaminated, needles and/or other disposable sharp implements. When the tool fails to remove the impaction, the user typically resorts to using her fingers or fingernails to complete the extraction. Using the comedo extractor also does nothing with regard to preventing formation of acne bacteria and future acne flare-ups.

Another example of a previous attempt to help acne patients is described in U.S. Patent Application 20080082111, entitled “Device and Method for the Evacuation of Matter Beneath the Skin”. The publication generally describes a device fashioned in the form of a forcep or tweezer with two opposing lateral arms, and another device having scissor-like handles. Because of the fixed position of the squeezing arms of the device, the device cannot grip acne related impactions on contoured surfaces of one's face that are prone to clogged skin pores, including areas around the curvature of the nose. It also appears unsuitable to use over the curved, and unstable surfaces of the tops of ears, the ear lobe, and where the ear joins the cheek. In addition, using the device is awkward and proper positioning of the device over the impaction is very problematic. Moreover, the rigid squeezing faces of the device, with fixed size and shape, causes bruising and scarring to tissue surrounding stubborn blockages. Furthermore, the device is not disposable, is easily contaminated and potentially infectious when used.

Given the many problems and disadvantages associated with conventional approaches to acne treatment, there is a need for improved methods for helping people afflicted with acne.

### SUMMARY OF THE INVENTION

The present invention relates to a finger-assisted device for hygienic extraction of acne related skin impactions, and methods and kits for hygienic extraction of acne related skin impaction.

In accordance with the present invention, the device includes a central portion; at least one tab radially disposed around said central portion; a top side; and a bottom side, said top side sized and configured for hygienic contact with impacted skin, said bottom side sized and configured for adhesive contact with a fingertip; and wherein said tab has a proximal end and a distal end, said proximal end integrally attached to said central portion, and said distal end sized and configured for pressure-assisted release of the contents of the impaction.

One object of the invention is to provide a device made from molded pulp, molded fiber, compacted organic cotton, rubber, plastic, cloth, sponge, foam substance, or any combination thereof.

Another object of the invention is to provide a device suffused with a substance selected from an oil, a natural additive, an anti-acne cream, an antimicrobial, or any combination thereof.

According to another object of the invention, the bottom side of the device includes a removable backing configured for removal prior to adhesive contact with the fingertip.

According to yet another object of the invention, the top side of the device includes at least one malleable groove preferably extending through the central portion to the distal end of the tab.

Another object of the invention is to provide a device that includes at least one malleable groove at a distal end of at least one tab.

Another object of the invention is to provide a device that includes a plurality of tabs radially disposed around a central portion, wherein the distal end of each tab is sized and configured for pressure-assisted release of the contents of a different sized impaction.

According to another object of the invention, the device has a thickness of about 1 mm to about 5 mm.

The present invention also relates to a method for hygienic extraction of an acne related skin impaction. In accordance with one object of the invention, a first device is adhered to a first fingertip; a second device is adhered to a second fingertip; the first and second devices are applied adjacent to the impacted skin; and the acne related impaction is extracted.

The present invention also relates to a kit for hygienic extraction of an acne related skin impaction. According to one object of the invention, the kit comprises a first device; a second device; and at least one substance capable of being suffused in the first and second device, wherein the first and second device each comprise a central portion; at least one tab radially disposed around said central portion; a top side; and a bottom side, said top side sized and configured for hygienic contact with the impacted skin, and said bottom side sized and configured for adhesive contact with a fingertip; and wherein said tab has a proximal end and a distal end, said proximal end integrally attached to said central portion, and said distal end sized and configured for pressure-assisted release of the contents of the impaction.

Yet another object of the invention is to provide a device in which the central portion is elevated in height relative to the height of at least one tab.

It is to be understood that both the foregoing general description and the following detailed description are exemplary and explanatory and are intended to provide further explanation of the invention as claimed. Other objects and features of the invention will become apparent from the following detailed description considered in conjunction with the accompanying drawings. The accompanying drawings are included solely for purposes of illustration and not as a definition of the limits of the invention. Also, the drawings are not drawn to scale, and are merely conceptual in disclosing the preferred embodiments of the invention.

### BRIEF DESCRIPTION OF THE DRAWINGS

FIG. 1 depicts a top-side view of a device, which includes a plurality of malleable grooves, according to one embodiment of the present invention;

FIG. 2 depicts a bottom-side view of a device, which includes a removable backing layer and a plurality of malleable grooves, according to another embodiment of the present invention;

FIG. 3 depicts another embodiment of bottom-side view of a device, which includes a removable backing layer and a malleable groove;

FIG. 4 depicts an embodiment of bottom-side view of a device, after removal of a removable backing layer;

FIG. 5 depicts a side view of one embodiment of a device, which includes an elevated central portion and a malleable groove, according to the present invention;

FIG. 6 depicts a schematic view of a user employing a two-handed technique for hygienic extraction of an acne-related skin impaction, according to one embodiment of the present invention; and

FIG. 7 depicts a schematic view of a user employing a one-handed technique for hygienic extraction of an acne-related skin impaction, according to another embodiment of the present invention.

### DETAILED DESCRIPTION OF THE PREFERRED EMBODIMENTS

As described in further detail herein, the present invention relates to a finger-assisted device for hygienic extraction of acne related skin impaction, and methods and kits for hygienic extraction of acne related skin impaction.

The device of the present invention is preferably a disposable, non-surgical, non-invasive device for hygienic extraction of an acne related skin impaction. The device is designed for safely drawing skin tissue underneath an impaction thereby lifting it so that the contents of the impaction may be easily removed. The device thus removes the contents from an acne related skin impaction, for instance, puss, sebum, blood, other fluid, bacteria, dirt, dead cells or dried or solid matter that may be present from acne pimples, whiteheads and blackheads. The present invention also offers surprising advantages in terms of preventing future acne flare-ups by killing acne bacteria.

The device of the present invention may be used by an individual afflicted with any acne related impaction. The term “acne related impaction” is intended to broadly encompass the comedones, pustules, pimples, whiteheads, blackheads and other abnormal conditions of the skin of an individual that are commonly associated with acne, acne vulgaris, and other skin conditions related to acne. Such impactions are frequently associated with inflammatory swelling and other symptoms. Other acne related impactions may be produced by changes in the pilosebaceous units, skin structures consisting of a hair follicle and its associated sebaceous gland via androgen stimulation. In certain instances, acne and acne-related impactions may be characterized by non-inflammatory follicular papules or comedones and/or by inflammatory papules, pustules, and nodules. Comedones may include enlarged hair follicles plugged with oil and bacteria. Other examples of acne-related impactions include “cysts” or large nodules which include, for instance, nodulocystic or severe cases of inflammatory acne. Any area of the skin on a person's body can be affected with acne-related impactions, including areas with dense populations of sebaceous follicles. Commonly affected areas include the face, the upper part of the chest, the neck, arms, legs, and the back. Severe acne is often inflammatory, but it can also be manifest in non-inflammatory forms. Acne lesions are also frequently associated with blemishes of the skin of an afflicted person.

The present invention can be used by a person of any age, and on any area of a person's body that is affected by the acne-related impactions described herein. Typical users of the device will include adolescents and young adults, since acne is frequently seen in these age populations.

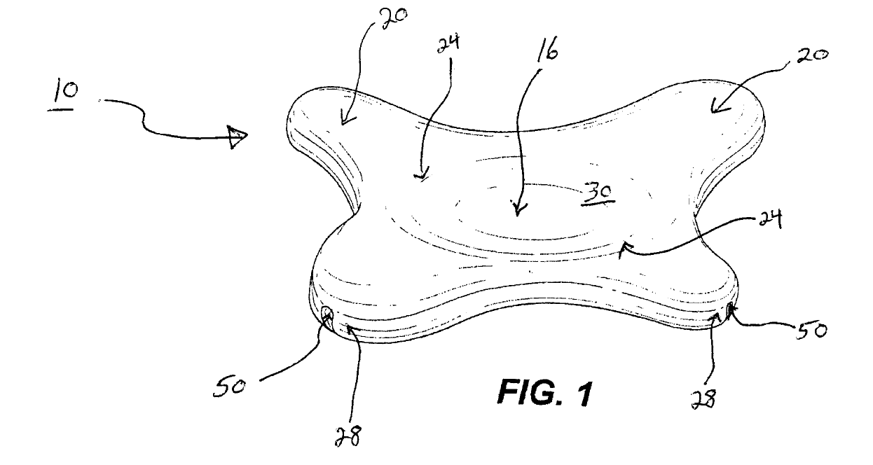

Referring to FIG. 1, one embodiment of the device 10 is shown. FIG. 1 shows a perspective view of the top side 30 of the device. The top side 30 of each device is sized and configured for hygienic contact with the impacted skin. The top side 30 of the device 10 is preferably formed from a material that provides sufficient traction for gripping the skin around the impaction 100, and for allowing a user to perform hygienic pressure-assisted release of the contents from the acne related impaction 100. The device 10 also includes at least one tab 20 radially disposed around a central portion 16. The number of tabs shown in FIG. 1 is for illustration purposes. It is to be understood that the device 10 can include a plurality of tabs 20, and thus can include any number of tabs 20, as needed or desired.

The tabs 20 which are radially disposed around the central portion 16 of the device 10 may be sized and configured for extraction of different size impactions. According to a preferred embodiment, the device 10 preferably includes a plurality of tabs 20 radially disposed around the central portion 16.

Referring again to FIG. 1, each of the tabs 20 has a proximal end 24 and a distal end 28. The proximal end 24 of each tab 20 is integrally attached to the central portion 16 of the device 10. The distal end 28 of each tab is sized and configured for pressure-assisted release of the contents of an impaction 100. The present invention also encompasses devices 10 with larger, padded disposable tabs 20.

According to another preferred embodiment of the invention, the device 10 also preferably includes at least one malleable groove 50. According to one embodiment of the present invention, the device 10 includes a plurality of malleable grooves 50.

The top side 30 of the device can optionally include at least one malleable groove 50 that extends through the central portion 16 to the distal end 28 of the tab 20 (not illustrated).

The device 10 may be made using any suitable material including, for example, 100% cotton fiber or compacted organic cotton. The device 10 may also be made using any other suitable material known in the art, for instance, molded pulp, molded fiber, rubber, plastic, gel, cloth, paper, paper mache, cotton, woven or non-woven fabrics, compacted fibrous material such as felt, feather, fur, thread, sponge, compacted tissue paper, a foam substance, soft, dense textile or synthetic material or any blend or combination thereof. For instance, the device 10 may be made using any combination of paper, paper mache, cotton, felt, and/or felt-like material. The material used to make the device 10 is preferably sterilized.

The device 10 is more preferably made from molded pulp, molded fiber, compacted organic cotton, rubber, plastic, cloth, sponge, a foam substance, or any combination thereof. In a preferred embodiment, the device 10 is made of a material that is capable of absorbing and/or adsorbing a liquid, cream or gel upon contact. The device 10 may be made using a substantially dry or anhydrous material, which is capable of absorbing or adsorbing moisture. A user of the present invention can apply, for example, an anti-acne liquid, cream or gel to the device 10 prior to adhering the device 10 to a fingertip 78 for extraction of the contents of an acne related impaction 100.

According to a preferred embodiment, the device 10 is uniformly soft to the touch while maintaining the stiffness characteristics of a compacted felt-like material. The device 10 may also have stiffer or less pliable characteristics as desired. For instance, the device 10 may have stiffer or less pliable characteristics at varying points along the length of the tabs 20 radially disposed around the central portion 16 of the device 10. In a preferred embodiment, the device 10 is constructed as a soft felt-like device 10 that may be used throughout a grooming session involving multiple manual extractions.

The device 10 may also be fabricated to have a desired stiffness to the bottom side 40 that gradually becomes more pliable throughout the skin-gripping surface of the top side 30 of the device 10. According to another embodiment, the device 10 may also include a two-ply material in which the bottom side 40 includes a relatively stiff layer and the top side 30 includes a material with a relatively softer texture.

The device 10 may be formed such that the device 10 has any suitable or desired size, shape, appearance, color and dimensions. The size of the device 10 is preferably large enough to be securely adhered to a fingertip 78 of a user. The central portion 16 of the device 10 may be generally concentric in shape, but it may also be substantially rectangular, square, round, oval, triangular, or any other geometric shape as desired.

In one embodiment, the device 10 is preferably circular in shape, and bears a plurality of tabs 20 that encircle and extend from the central portion 16. The device 10 may also take any other shape such as an “I”, oval, quadrilateral, star, flower or any shape and configuration having a multi-pointed design of the same size with or without notches or appendages that protrude from the central portion 16. The device 10 may also have a generally round or generally square shape. The device 10 is preferably sized to accommodate the size of a normal human fingertip 78. In one example, the device preferably measures about 1 and ⅛ inch by 1 inch. The skin-gripping surface of the top side 30 of the device 10 may, in one example, generally have the shape of a parabola. The device 10 may also be fabricated as a pre-moistened or pre-medicated pad with one or more tabs 20, including a plurality of tabs 20.

The device 10 may also be fabricated such that it has any color or multi-color appearance. The device 10 may therefore be suffused, covered with, or impregnated with one or more colorants or dyes. Designs, markings, or other features may also be embedded, e.g., for aesthetic purposes, on any part of the device 10. The bottom side 40, top side 30, or both sides of the device 10 may include designs, patterns or other artistic features. The device 10 may also be scented with one or more fragrances or other scenting agents.

The device 10 may also have any suitable thickness, as needed or desired. According to a preferred embodiment, the device has a thickness of about 1 mm to about 5 mm. In a preferred embodiment, the top side 30 of the device 10 has a generally concave shape when viewed in cross-sectional view.

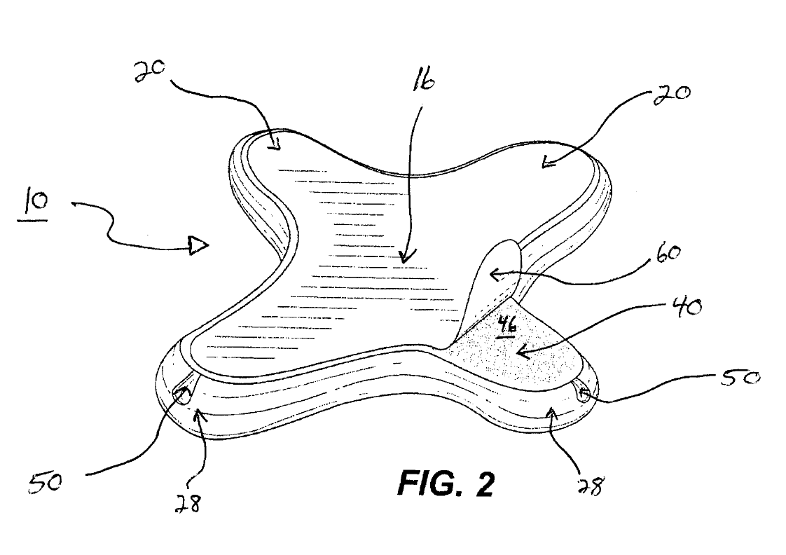

Referring to FIG. 2, according to another embodiment, the device 10 preferably includes at least one malleable groove 50 at a distal end 28 of at least one tab 20. When the device 10 has been positioned by a user, such that the surrounding tissue is compacted prior to extraction of the contents from the impaction 100, the pliable material that forms the malleable groove 50 responds to the increasing pressure during this stage of the extraction. With the targeted application of pressure to each tab 20 during the extraction, the malleable groove 50 functions to entrap and collect the contents that are extracted from the impaction 100. The grooves 50 thus surprisingly help to target and trap the contents during an extraction. Also, during the extraction, the contents of the impaction 100 are thus safely delivered to the skin's surface.

It is to be understood that the embodiments shown in the figures are only for illustrative purposes, and that the particular shape, size and dimensions of the tabs 20, distal ends 28 of the tabs 20, and the malleable groove 50s may be configured as needed or desired.

Referring again to FIG. 2, according to one embodiment of the present invention, a perspective view of the bottom side 40 of the device 10 is shown with a plurality of malleable grooves 50 at the distal ends 28 of the tabs 20.

As described in further detail below, the bottom side 40 of the device 10 is sized and configured for adhesive contact with a fingertip 78. The term “adhesive contact” is meant to broadly encompass any suitable material or combination of materials, and any suitable method, which functions to removably adhere or attach the bottom side of the device 40 to a fingertip 78. The adhesive contact is intended to be reversible, such that the user can remove the bottom side 40 of the device 10 from the fingertip 78 after the device 10 has been used for an extraction procedure.

To achieve the adhesive contact with the fingertip 78, the bottom side 40 of the device 10 preferably includes one or more suitable adhesive materials, which enables a person to adhere the device to the user's fingertip 78 while it is used for an extraction, and during as many grooming sessions as the user desires. “Grooming session” is intended to refer to a session or period of time in which a user performs extraction of an acne-related impaction 100. According to one example, for providing adhesive contact with a fingertip 78, the bottom side 40 of the device 10 can be coated with any suitable adhesive substance, such as an adhesive gum, to provide an adhesive surface 46. Preferably, the adhesive gum has low-tack characteristics and is preferably a hypoallergenic all-natural non-sensitizing adhesive-gum coating product. Any suitable type of adhesive substance may be used in accordance with the present invention. Representative examples include adhesive substances of plant origin, such as plant gums, and other gums having considerable adhesive characteristics. Examples include, but are not limited to, gum Arabic and gum tragacanth. One or more synthetic adhesive materials may also be used to form an adhesive surface 46. Elastomers, thermoplastics, emulsion, and thermosetting adhesives based on polyvinyl acetate, epoxy, polyurethane, cyanoacrylate polymers are representative examples of synthetic adhesives that may be used.

Again referring to FIG. 2, according to a preferred embodiment, the bottom side 40 of the device 10 preferably includes a removable backing layer 60 configured for removal prior to adhesive contact with a fingertip 78. The adhesive surface 46 of the bottom side 40 of the device 10 is thus revealed by removing such a protective, removable backing layer 60. Examples of suitable materials for the removable backing layer 60 include, for instance, natural or recycled non-stick paper-backing materials, cloth, plastic or rubber materials, and other suitable materials.

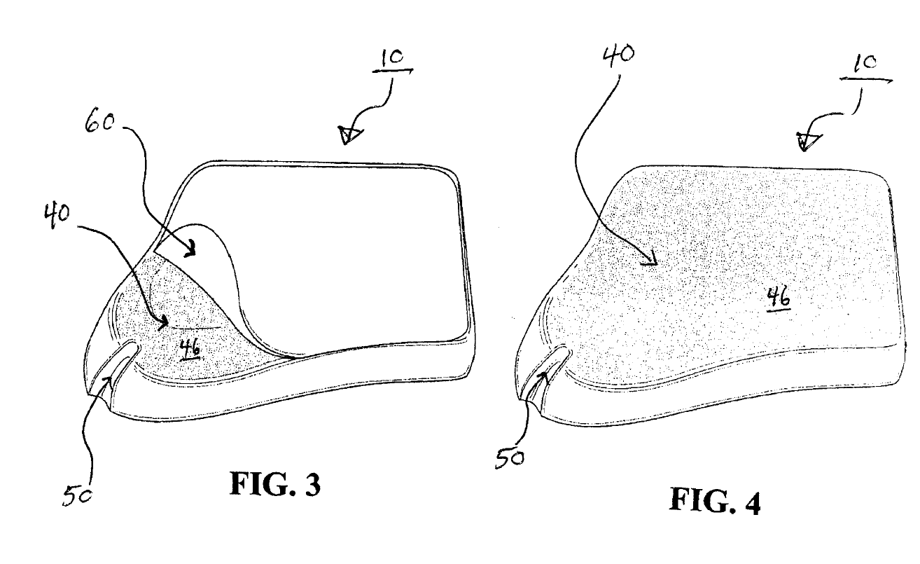

Referring to FIG. 3, another embodiment of a device 10 is shown, which depicts a different shape and size of the device 10 for illustrative purposes. The bottom side 40 of the device 10 is shown, along with an adhesive surface 46, removable backing layer 60 and malleable groove 50. FIG. 4 depicts another bottom-side view of a device 10, after removal of the removable backing layer 60, thus exposing the adhesive surface 46.

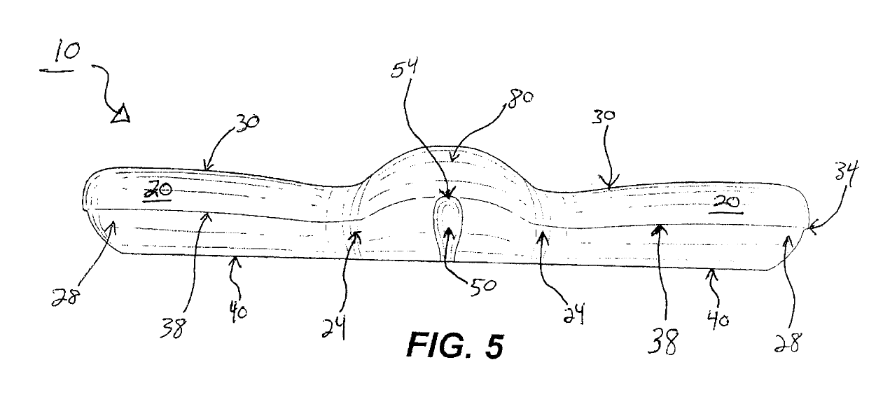

FIG. 5 depicts a side-perspective view of one preferred embodiment of the present invention. The device 10 includes an elevated central portion 80. The device 10 may optionally include a plurality of malleable grooves 50, which can be sized, shaped and configured as needed or desired. Referring to FIG. 5, the distal end 28 of each tab 20 is preferably formed such that there is a slight protrusion 34 of each distal end 28. The protrusion 34 is preferably located just above the horizontal center line 38 which separates the top side 30 and bottom side 40 of each tab 20. As shown from the side-perspective view in FIG. 5, the protrusion 34 at the distal end 28 is preferably nearer the top side 30 of each tab 20. During hygienic extraction of an acne related skin impaction, it has been found that the protrusion 34 assists with supporting and purging trapped matter from beneath the skin.

The upper edge 54 of each malleable groove 50 is preferably aligned with the horizontal center line 38 separating the top side 30 and bottom side 40 of each tab 20. However, it is to be understood that the embodiment shown in FIG. 5 is only for illustrative purposes, and that the particular shape, size and dimensions of the elevated central portion 80, the tabs 20, distal ends 28, protrusion 34 and malleable grooves 50 may be configured as needed or desired.

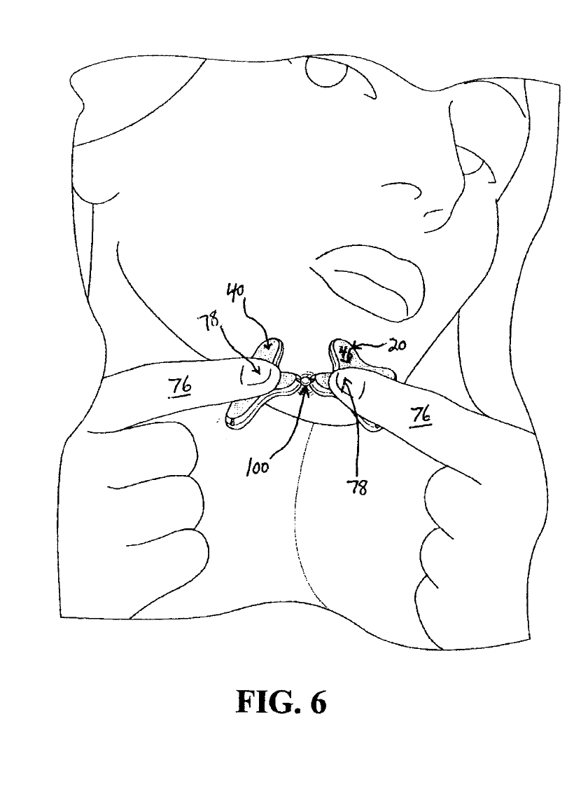

As described herein, the present invention also relates to methods for hygienic extraction of an acne related impaction 100. Referring to FIG. 6, according to one embodiment of the present invention, a schematic view is shown of a user employing fingers 76 of both hands for hygienic extraction of an acne-related impaction 100. A first device 10 is adhered to a first fingertip 78, and a second device 10 is adhered to a second fingertip 78. The first and second devices 10 are applied to the impacted skin and the acne-related impaction 100 is hygienically extracted. When a user finishes an extraction process for removing the contents from the acne related impaction 100, the devices 10 of the present invention can be safely discarded.

It is to be understood that the term “finger” as described herein, according to the present invention, is intended to encompass not only a user's index finger but also each of the other fingers on each hand, including a user's thumb. Therefore, in one embodiment, a user may use one or both of his or her thumbs for hygienic extraction of an acne-related impaction, as described herein. Similarly, the term “fingertip” as described herein is intended to include not only the tip of one's index finger or other fingers, but also the tip of a user's thumb.

In a preferred manner, a user affixes the first and second devices 10 to the underside of his/her index fingertips 78. While offering protection from fingernails and providing traction to avoid slipping over oily or moist skin, the devices 10 are positioned to surround the outer edges of the impaction 100. The present invention thus provides sufficient traction over the skin which enables a person, with both of his/her fingers 76 and fingertips 78, to compress from each side and slightly beneath the impaction 100, causing a firm base of underlying skin tissue to accumulate, forming a bed beneath the impaction 100, thus producing hygienic extraction of the contents from the impaction 100. This compressing motion is desired throughout the extraction procedure, since it also provides unexpected control of the devices 10 throughout the extraction process which is especially useful over slippery, oily skin.

Referring again to FIG. 6, the adhesive surface 46 on the bottom side 40 of the device 10 allows a person to adhere a pair of devices 10 precisely into place on his/her left and right index fingertips 78 which also aids in positioning the devices 10 around the impaction 100. The adhesive surface 46 also allows the user to reposition the pair of devices 10 on the underside of her/his fingertips 78 repeatedly as many times as needed or desired before the user determines that the usefulness of the devices 10 has expired, at which time the devices 10 may be discarded.

A pair of devices 10 can thus be adhered to the underside of each of two fingertips 78 of the index fingers, under most circumstances, but may also be adhered to other fingers to aid in the removal of acne related impactions 100. The device 10 can also be affixed to the fingers of a second person wishing to remove the acne related impactions of another.

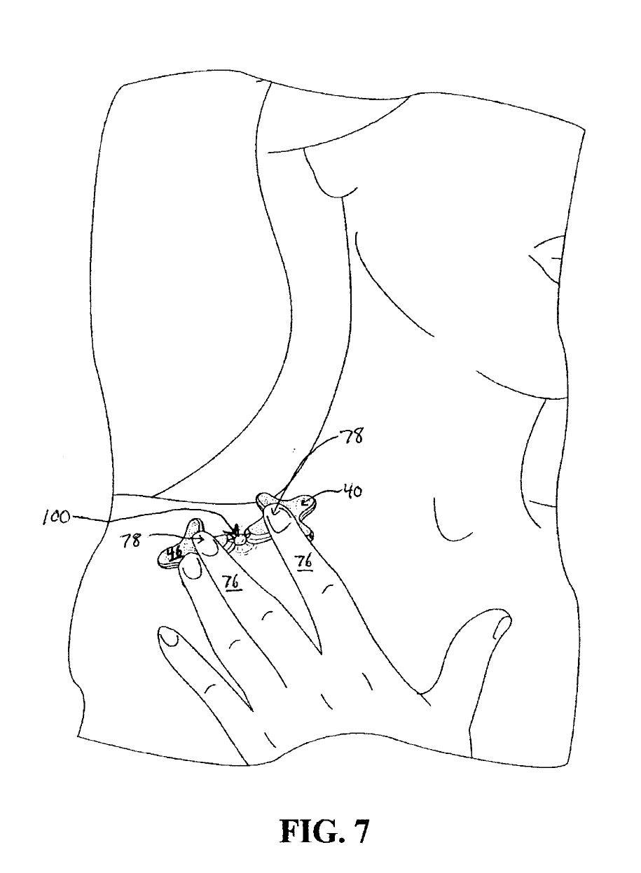

Referring to FIG. 7, a schematic view is shown of a user employing a one-handed technique for hygienic extraction of an acne-related impaction 100, according to another embodiment of the present invention. A first device 10 is adhered to the underside of one fingertip 78, and a second device 10 is adhered to the underside of another fingertip 78 of the same hand, thus allowing the user to perform the one-handed hygienic extraction. Also, as depicted in FIG. 7, the adhesive surface 46 allows the user to access impactions 100 of the skin located on areas of the body that are not as easily accessed. The adhesive surface 46 of the devices 10 surprisingly and unexpectedly provides traction upon oily or moist skin which is common around the site of an acne related impaction 100.

As described herein, the size, shape and configuration of the tabs 20 of the device 10 may be configured as needed. The selection of the desired size and shape tabs 20 thus allows the user to concentrate the pressure exerted by the user's fingers 76 and fingertips 78 around the impaction 100. According to a preferred method of hygienic extraction, once the devices 10 are adhered to the fingertips 78, the user applies a gentle downward and inward motion to each device 10 around the impaction 100. This pressure-assisted motion thoroughly encompasses and supports the impaction 100, such that the pressure that is applied functions to express or release the contents from the impaction 100, and to safely lift the contents of the impaction 100 to the surface of the user's skin. In this manner, the contents of the impaction 100 are safely lifted by a gently applied downward and inward pressure that carefully supports the impaction 100 before the contents are ejected from the impaction 100. It has been found that the tabs 20 of the present invention, along with a proper pressing or compression technique as described herein, eliminates the need to lance or puncture the skin which is a common and serious problem associated with conventional techniques for acne removal.

The device 10 also provides safe removal of contents from an impaction 100 by manipulating skin tissue to lift the impaction 100, while the tabs 20 provide the advantage of being deformable, malleable and flexible. Accordingly, the tabs 20 can easily change shape during the extraction procedure, allowing the user to carefully control the movement of the contents of the impaction 100 as they are expressed to the surface of the skin.

According to another preferred embodiment, a substance selected from an oil, a natural additive, an anti-acne substance, an antimicrobial, herbal component, a vitamin, or any combination thereof can be partially or completely suffused, impregnated, infused, absorbed, and/or adsorbed within and/or onto the device 10. The oil, natural additive, anti-acne substance, antimicrobial, herbal component, vitamin, or any combination thereof may be formulated and applied in any acceptable form including, for example, as a lotion, cream, gel, solution, or suspension for topical application.

According to a preferred embodiment, the device 10 is infused or instilled with one or more suitable natural or organic additives or excipients; anti-acne creams or lotions; anti-inflammatory agents, and/or antimicrobial or other therapeutic (e.g., analgesic) agents. Such agents are preferably released from the device 10 to the site of the impaction 100 while the device 10 is being used for removing the contents from the impaction 100. The agents can be partially or completely absorbed and/or adsorbed throughout the device 10. The present invention thus surprisingly and unexpectedly helps to reduce or eliminate the bacteria and inflammation associated with acne flare-ups. Examples of suitable anti-acne additives are those that contain all-natural botanical extracts and/or natural oils, and are deemed safe and non-toxic. The natural ingredients that can be used include those that help reduce or eliminate acne blemishes, pimples, and blackheads and even prevent acne breakouts to reoccur. The present invention also encompasses devices infused with other medications that are deemed safe and effective for application to the skin.

Examples of natural botanical extracts (including extracts of fruits, vegetables, spices, herbs, etc.) include, for example, extracts from sedge plants (Carex distachva) that contain antioxidants, meristem extract; vanilla extract, quillaia extract, mate leaf extract, coffee extract, cocoa extract, chicory root extract, rose hip extract, fenugreek seed extract, green tea leaf extract and carob extract. Other examples include medicinal plant extracts. These are merely provided as representative examples and are in no way meant to limit the scope of the invention.

Examples of natural oils include, but are not limited to, frankincense essential oil and neroli oil. Other examples include jojoba oil, eucalyptus oil (for instance, from eucalyptus globulus), oil of anise, oil of sandalwood, oil of pure geranium, oil of jasmine, oil of pure peppermint, oil of melissa, oil of ginger and other pure essential oils. Still other examples include sesame oil, garlic oil, parsley leaf oil, cinnamon bark oil and cubeb oil. These are merely provided as representative examples and are in no way meant to limit the scope of the invention.

Examples of suitable antimicrobial agents include, for instance, topical antibiotics such as fusidic acid; bacitracin; mupirocin; and retapamulin. Other therapeutic agents that can be used include, for example, benzoyl peroxide, collagenic palmitoyl acid, bisabolol; and other therapeutic agents, for instance, anti-inflammatory and/or analgesic agents. These are merely provided as representative examples and are in no way meant to limit the scope of the invention.

Other types of natural extracts, pure essential oils, and other suitable natural or organic additive or excipients; anti-acne creams or lotions; anti-inflammatory agents, and/or antimicrobial or other therapeutic (e.g., analgesic) agents can also be used in accordance with the present invention. The specific examples described herein are for illustrative purposes only, and are not meant to limit in any way the scope of the invention.

The safe and effective amounts and percentages of each additive or agent to be suffused, infused, or partially or completely absorbed and/or adsorbed throughout the device 10 can be readily determined by a person of ordinary skill in the art without undue experimentation. Such determination of safe and effective amounts can be readily determined by those skilled in the art of treating acne and other skin conditions. Moreover, the amounts and percentages of each additive or agent can vary depending on a number of factors, including the severity of the acne of a particular individual, and the amounts used can be adjusted as needed or desired. The present invention can thus be used to administer an antimicrobial and/or other agent so that while in use, it can help eliminate the bacterial strain associated with acne, thereby potentially reducing future inflammation and infection in adjacent pores that causes the formation of pimples. When treating acne in accordance with the present invention, maintaining proper hygiene is critical. Skin tissue is already aggravated and vulnerable to further secondary infection as a result of open and exposed lesions. A user may therefore cleanse acne prone areas with an all natural antibacterial soap after application of the device 10 of the present invention to further provide for rapid elimination of acne.

The present invention also relates to a kit for hygienic extraction of an acne related impaction 100. According to one object of the invention, the kit includes a first device 10 and a second device 10, and at least one substance capable of being suffused in the first and second devices 10. The first and second devices 10 each comprise a central portion 16, at least one tab 20 radially disposed around the central portion 16, a top side 30 and a bottom side 40 as described in further detail herein. Also, as described herein, the top side 30 is sized and configured for hygienic contact with the impacted skin, and the bottom side 40 is sized and configured for adhesive contact with a fingertip 78. Each tab 20 has a proximal end 24 and a distal end 28. The proximal end 24 is integrally attached to the central portion 16, and the distal end 28 is sized and configured for pressure-assisted release of the contents of the impaction 100.

Each of the devices 10 may be designed to extract any sized impaction 100. A user can thus select from devices 10 having several uniquely sized and shaped tabs 20, which are integrally connected to the central portion 16 of the device 10. The tabs 20 are appropriately shaped and sized for each extraction. The tabs 20 may be used throughout a grooming session involving multiple manual extractions. When finished, the devices 10 are safely discarded.

According to a preferred embodiment, the kit includes a pair of devices 10 which are mounted on a disposable card or backing material, such as cardboard (not shown). A user can simply remove the devices 10 from one or more disposable cards or backing materials, for use in accordance with the present invention. The disposable card or backing material can also function to protect the top side 30, bottom side 40 and tabs 20 of the device 10 from breakage or damage during packaging and transport. Multiple devices 10 can also be dispensed in a single kit by contacting the devices to a protective non-stick card that is coated with a non-stick film. The non-stick film protects the devices 10 by covering them until the devices are removed for their use.

According to another preferred embodiment, the kit can include a plurality of devices 10 packaged in any other suitable and convenient manner. For instance, the kit can include a coiled strip that comprises a row of devices 10 adhered to the strip (not shown). The coiled strip is preferably fed through a slit-opening from a box. The devices 10 can also be sandwiched between two layers, for instance, with the adhesive side of each device 10 facing a layer of backing paper. A thin continuous strip of peel-away film can also be mounted over the devices 10 to give a sanitary-sealed appearance. There can also be perforations between every two devices 10 along the strip for easy tear-off and removal.

According to another embodiment, the kit includes a small box that comprises a plurality of devices 10 mounted in pairs and individually dispensed on small sheets of non-stick backing paper. One sheet containing two devices 10 may be exposed through a top opening of the box in a continuously fed “pop-up” system. The sheets may be fabricated as a connected strip of sheets. Packaging preferably involves inserting the connected strip of paper sheets (with the devices 10 applied to each of the sheets) into the box after the strip of sheets have been stacked by folding them along their separating perforations in “slinky-like” fashion. As the sheets are dispensed from the box, one pair follows the other through the top opening of the box and each pair is torn off along the perforations for use.

Additionally, the device 10 of the present invention, which can be used by a person grooming themself of acne related impactions 100 of the skin in the privacy of their own home, can also be used in a clinic or hospital by professional clinicians on their patients to remove the contents from an acne related impaction 100. The device 10 can also be used to remove more than one impaction such that multiple acne-related impactions can be expressed simultaneously.

As further described herein, the present invention provides several unexpected advantages over conventional approaches for acne removal. The present invention has been unexpectedly found to shield the skin from fingernail damage and provide traction to avoid slipping over oily or moist skin. Also, no lancing or puncturing of the skin prior to extraction is required. The present invention enables a user to cleanly, safely and efficiently extract unsightly acne pimples, whiteheads and blackheads. Extraction using the present invention speeds healing another surprising benefit of the present invention is the reduction and even elimination of scarring that typically occurs when someone attempts to remove an impaction of the skin without the aid of the current invention. In addition, the device of the present invention also helps to avoid further inflammation to the affected area caused by abrasions that are typically caused by loosing traction and “skidding” across the skin. Also, when the device of the present invention is suffused with an antimicrobial, it can help eliminate the bacterial strain associated with acne, thereby reducing future inflammation in adjacent pores that causes the formation of pimples.

The foregoing descriptions of specific embodiments are provided by way of the following examples of the present invention. These examples are meant to illustrate certain embodiments of the invention, but in no way limit the scope of the present invention. It is foreseeable that various changes, combinations and modifications thereof will become apparent to those skilled in the art by this enabling disclosure.

### EXAMPLES

### Example 1

A user may select from among devices 10 having any desired sized and shaped tabs 20 that extend radially from the central portion 16 of the device 10 depending upon the size, type and/or location of the impaction to be removed. One particular device is constructed with a plurality of tabs, in which the distal end of each tab has a malleable groove and the distal end is approximately 3/16 inches in diameter in width when measured from one side of the distal end of the tab to the other side. The malleable grooves which are located at the distal ends of each tab provide for safe and effective removal of the contents of the impaction during the hygienic extraction. This sized tab is surprisingly effective in the removal of small-diameter impactions 4 mm in diameter and under, over areas of the face or body covered by a thinner layer of skin tissue that is backed by hard flat bone structure where impactions may be easy to extract, as they are often located closer to the skins surface. Examples include impactions clogging the skin located on the forehead, brow, cheek bone and chin area of the face. This sized device can also surprisingly aid in more precise placement while a user reaches specific impactions on parts of the face, ears and other areas. This device is also particularly effective along the bony surfaces of the arms, legs and along shoulders and collar bone. This device can also be particularly effective for hygienic extraction of impactions over the especially oily and angles and curves located on the nose, where the nose meets the cheek, ear meets the face, surrounding the lips, and any other portion of the body where the underlying tissue compacting ability helps raise an impaction so that it may be easily drawn from the skin.

### Example 2

A device is constructed with a plurality of tabs, in which the distal end of each tab has a malleable groove and the distal end is approximately ¼ inches in diameter in width when measured from one side of the distal end of the tab to the other side. The malleable grooves which are located at the distal ends of each tab surprisingly provide for safe and effective removal of the contents of the impaction during the hygienic extraction. This device offers many benefits for removal of acne related impactions, in particular, for removal of larger impactions up to approximately 6 mm in diameter, while offering a broader range of impaction removal capabilities that can be effective nearly everywhere on a persons skin.

### Example 3

A device is constructed with a plurality of tabs, in which the distal end of each tab has a malleable groove and the distal end is approximately ⅜ inches in diameter in width when measured from one side of the distal end of the tab to the other side. This device is particularly effective in removal of larger acne-related impactions of up to approximately 9 mm in diameter. The malleable grooves which are located at the distal ends of each tab surprisingly provide for safe and effective removal of the contents of the impaction during the hygienic extraction. The wide diameter of the distal end provides greater traction and enhances accuracy that is required during extractions when it is difficult for the user to see the exact placement of the device around the impaction. These areas may include extractions performed behind the neck, the area of the lower back, and on the back of the thigh. Other areas of the body that may benefit using this device include regions of the body located on or over the tops of each shoulder, the back of the upper arms, within and around the under-arms, and on the back.

### Example 4

The device is fabricated from molded pulp, as a soft, adhesive-backed device that is used throughout a grooming session involving multiple manual extractions. The adhesive layer on the bottom side of the device adheres to an index finger, and the device provides traction upon oily or moist skin. The devices are used in pairs, such that one device is adhered to the underside of the fingertip of the user's left index finger, and the other device is adhered to the underside of the fingertip of the user's right index finger. Each felt-like device is readily adjustable to extract any sized impaction. Each device is appropriately shaped and sized for the extraction, depending on the size of the impaction. The selection of the device with desired shaped tabs allows the user to concentrate the pressure exerted around the impaction. A malleable groove is located at the distal end of each tab. A gentle downward and inward motion thoroughly encompasses and supports the impaction, thereby providing the energy and pressure that allows for easy and effective ejection of the contained sebum and puss to the surface of the skins natural pore. The contents of the impaction are safely removed, and the malleable grooves at the distal ends of each tab assist with capturing the contents of the impaction during the extraction. The specially designed tabs are positioned around the outer edge of the impaction to enable it to be trapped then lifted by a very gently applied-downward and inward pressure that carefully supports the impaction before the contents from the impaction are ejected. The specially designed tabs help alleviate the need to lance or puncture the skin beforehand. The device is additionally suffused with an antimicrobial so that while in use, it helps eliminate the bacteria associated with acne, and reduces future acne flare-ups.

What is claimed is:

1. A finger-assisted device for hygienic extraction of an acne related skin impaction, comprising:
a central portion;
at least one tab radially disposed around said central portion;
a top side; and
a bottom side,
said top side sized and configured for hygienic contact with the impacted skin, and said bottom side sized and configured for adhesive contact with a fingertip; and
wherein said tab has a proximal end and a distal end, said proximal end integrally attached to said central portion, and said distal end sized and configured for pressure-assisted release of the contents of the impaction.

2. The device of claim 1, wherein the device is made from a material selected from the group consisting of molded pulp, molded fiber, compacted organic cotton, rubber, plastic, cloth, sponge, a foam substance, or any combination thereof

3. The device of claim 1, wherein the device is suffused with a substance selected from an oil, a natural additive, an anti-acne cream, a vitamin, an antimicrobial, or any combination thereof.

4. The device of claim 1, wherein the bottom side comprises a removable backing layer configured for removal prior to adhesive contact with the fingertip.

5. The device of claim 1, wherein the device further comprises at least one malleable groove at a distal end of said at least one tab.

6. The device of claim 1, wherein the top side comprises at least one malleable groove extending through the central portion to the distal end of the tab.

7. The device of claim 1, wherein the device comprises a plurality of tabs radially disposed around said central portion, wherein the distal end of each tab is sized and configured for pressure-assisted release of the contents of a different sized impaction.

8. The device of claim 1, wherein the central portion is elevated in height relative to the height of said at least one tab.

9. A method for hygienic extraction of an acne related skin impaction, comprising:
adhering a first device to a first fingertip;
adhering a second device to a second fingertip;
applying the first and second device to the impacted skin; and
extracting the acne related impaction,
wherein said first device or said second device each comprise:
a central portion;
at least one tab radially disposed around said central portion;
a top side; and
a bottom side,
said top side sized and configured for hygienic contact with the impacted skin, and said bottom side sized and configured for adhesive contact with the first or second fingertip.

10. The method of claim 10, wherein the first and second device are each made from a material selected from the group consisting of molded pulp, molded fiber, compacted organic cotton, rubber, plastic, cloth, sponge, a foam substance, or any combination thereof.

11. The method of claim 10, wherein the first and second device are suffused with a substance selected from the group consisting of an oil, a natural additive, an anti-acne cream, an antimicrobial, or any combination thereof.

12. The method of claim 10, wherein the bottom side of each of the first and second device comprises a removable backing layer configured for removal prior to adhesive contact with the first and second fingertip.

13. The method of claim 10, wherein the first device and second device each further comprise at least one malleable groove at a distal end of said at least one tab.

14. The method of claim 10, wherein the top side of each of the first and second device comprises at least one malleable groove extending through the central portion to the distal end of the tab.

15. The method of claim 10, wherein the first and second device each comprise a plurality of tabs radially disposed around said central portion, wherein the distal end of each tab is sized and configured for pressure-assisted release of the contents of a different sized impaction.

16. A kit for hygienic extraction of an acne related skin impaction, comprising:
a first device;
a second device; and
at least one substance capable of being suffused in the first and second device,
wherein the first and second device each comprise:
a central portion;
at least one tab radially disposed around said central portion;
a top side; and
a bottom side,
said top side sized and configured for hygienic contact with the impacted skin, and said bottom side sized and configured for adhesive contact with a fingertip; and
wherein said tab has a proximal end and a distal end, said proximal end integrally attached to said central portion, and said distal end sized and configured for pressure-assisted release of the contents of the impaction.

17. The kit of claim 18, wherein the first and second device are each made from a material selected from the group consisting of molded pulp, molded fiber, compacted organic cotton, rubber, plastic, cloth, sponge, a foam substance, or any combination thereof.

18. The kit of claim 18, wherein the substance is selected from the group consisting of an oil, a natural additive, an anti-acne cream, an antimicrobial, or any combination thereof.

19. The kit of claim 18, wherein the bottom side of each of the first and second device comprises a removable backing layer configured for removal prior to adhesive contact with the fingertip.

20. The kit of claim 18, wherein each of the first and second devices further comprises at least one malleable groove at a distal end of said at least one tab.

21. The kit of claim 18, wherein the top side of each of the first and second devices comprises at least one malleable groove extending through the central portion to the distal end of the tab.

22. The kit of claim 18, wherein the first and second device each comprises a plurality of tabs radially disposed around said central portion, wherein the distal end of each tab is sized and configured for pressure-assisted release of the contents of a different sized impaction.
